(19) United States
(12) Patent Application Publication
(10) Pub. No.: US 2021/0386793 A1
(43) Pub. Date: Dec. 16, 2021

(54) NEW MEDICAL USE OF OXALATE-REDUCING BACTERIA

(71) Applicant: OXTHERA INTELLECTUAL PROPERTY AB, Stockholm (SE)

(72) Inventors: Elisabeth LINDNER, Stockholm (SE); Bastian DEHMEL, Buonas (CH); Maria ÅKERMAN, Sollentuna (SE)

(73) Assignee: OXTHERA INTELLECTUAL PROPERTY AB, Stockholm (SE)

(21) Appl. No.: 17/282,439

(22) PCT Filed: Oct. 4, 2019
(86) PCT No.: PCT/SE2019/050970

§ 371 Date: Apr. 2, 2021

(30) Foreign Application Priority Data
Oct. 4, 2018 (EP) 18198742.1

(51) Int. Cl. A61K 35/74; A61P 13/12; A61P 9/00; A61K 9/48

(52) U.S. Cl. CPC A61K 35/74; A61P 13/12; A61K 9/4891; A61K 9/4858; A61P 9/00

(57) ABSTRACT

The present invention relates to the treatment or prevention of oxalate-related disorders, more particularly to the use of a pharmaceutical composition comprising Oxalobacter formigenes in the treatment or prevention of systemic oxalosis with cardiac involvement.

### TECHNICAL FIELD

The present invention relates to the field of oxalate-related disorders, and more particularly to the field of oxalate-reducing bacteria useful for the treatment of such disorders.

### BACKGROUND OF THE INVENTION

Primary hyperoxaluria (PH) type I, II and III are rare autosomal recessive inborn errors of glyoxylate metabolism, caused by various deficiencies in enzyme function, which lead to the overproduction of oxalate. Oxalate cannot be metabolised by human cells and is primarily eliminated through the kidneys and the gastrointestinal tract. High oxalate concentration, primarily in the form of calcium-oxalate crystals, damage the renal parenchymal cells.

As a result of disease progression, crystals are deposited in the kidney and then, as a result of disease progression, in the bone, in joints, in the cardiovascular room and in epithelium.

In addition to calcium-oxalate deposition, the condition is associated with inflammation and interstitial fibrosis (Cochat P. et al., N Engl J Med 369, 649-658, 2013, Hoppe B, Nature reviews Nephrology, 8, 467-475, 2012) in epithelium including the heart myocardium. In PH, marked hyperoxaluria is present from birth. The majority of the patients are symptomatic during childhood and are diagnosed before 10 years of age. In some cases, however, the disease may go unrecognised until patients reach 30-60 years of age.

PH can develop into chronic kidney disease, subsequently leading to End Stage Renal Disease (ESRD, chronic kidney disease (CKD) stage 5). These conditions are in general associated with kidney failure and a need for dialysis and transplantation. Typically, there is no cure for late stage CKD, but instead different treatments are used to alleviate the symptoms associated therewith. Systemic oxalosis is a condition involving deposition of oxalate in the kidney and in extra-renal tissues including the heart leading to a systemic involvement of oxalate in the body. Currently, only intensive dialysis is available to remove oxalate to improve the possibility for, and the outcome of transplantation.

Cardiovascular disease (CVD) is the leading cause of death in patients with CKD. Notably, there is a graded increased risk in mortality following any cardiac event including myocardial infarction and heart failure across the different stages of CKD. Appropriate tools for detecting early changes in cardiac function may facilitate determining the risk for CVD in CKD (Krishnasamy et al., Neprol Dial Transplant (2014) 29:1218-1225).

Deterioration of cardiac function can be caused by several reasons, such as disturbance of electrical pulses (heart rhythm), occlusion (pump volume and pressure) and deposition (contractability), among others.

Occlusion may occur from atherosclerosis, or other reasons, many of them including fat and/or cholesterol building up in vessel walls and heart tissue causing inflammation and reduction of the void volume. These diseases can lead to lower blood flow and complete occlusion of peripheral vessels and to brain hemorrhage due to high blood pressure.

Deposition and calcification may be caused by calcium oxalate or by calcium phosphate precipitating in tissue and vessel walls. This crystallization is calcifying tissue and causes stiffness affecting the contractability of the heart and the arterial vessels causing a worsening of systolic function.

Currently, ejection fraction (EF) measured from conventional transthoracic echocardiogram is the standard method for assessment of left ventricular (LV) systolic function. A reduced EF in a subject informs of global systolic dysfunction, indicating a risk for heart failure.

Recently, speckle-tracking echocardiography (STE) has emerged as a new imaging technology for measuring disturbances of the contractability of the heart muscle in LV systolic function. STE overcomes some of the intrinsic limitations of conventional echocardiograms in the assessment of complex LV myocardial mechanics and deformation parameters. In particular Global Longitudinal Strain (GLS) is a marker for early detection of changes in the LV contractibility (Lagies et al. Echocardiography, 2015 August; 32(8), 1250-60).

CVD, as the leading cause of death in patients with advanced stage kidney disease, can be due to the accumulation of oxalate crystals in the blood vessel epithelium and the myocardium, where high burden of oxalate crystals results in a stiffness decreasing the elasticity and hence the pumping capacity for the heart and vessels. High or rising concentrations of total plasma oxalate, i.e. total oxalate present in plasma including crystals bound by large molecules such as albumin, and soluble or free plasma oxalate, i.e. dissolved freely circulating oxalate, are indicative of advanced progression towards ESRD or that a patient is in ESRD.

The invention presented herein addresses the medical need for treatments for patients suffering from systemic oxalosis with cardiac involvement. In particular, a treatment capable of reversing the stiffness of the heart muscle (also referred to herein as the cardiac muscle or myocardium) and the decrease in heart function caused by oxalate crystals, as further explained herein, is introduced.

To date, dialysis of PH patients suffering from ESRD has not been shown to overcome the problems of excess oxalate production and deposition of oxalate crystals at renal and extra-renal sites (i.e. systemic oxalosis) (Costello et al., JASN, 1991 (1) 1289-1298). Even at very intense dialysis regimens (6-7 days/week), oxalate production is generally too high for dialysis to sufficiently reduce the oxalate concentration in blood and clear the body and tissues from accumulated oxalate crystal deposits. The high and rising concentrations of oxalate eventually result in decreased cardiac contractibility. A medical treatment, which in addition to dialysis, enhances or contributes to the removal of oxalate crystals, and consequently has a beneficial effect on the heart muscle function, would be of immense importance. Therefore, there is still a need in the art to identify such treatments.

Oxalobacter formigenes is a strict anaerobic bacterium that relies exclusively on oxalate as a substrate to obtain energy for its survival and growth. It is currently believed to be the most efficient oxalate-reducing enzymatic system that operates at neutral pH.

Administration of Oxalobacter formigenes to a subject in need thereof has been shown to have an effect on dietary oxalate absorption, but it has also been shown to have effect on the excretion of oxalate from plasma to the intestine, promoting the natural intestinal oxalate excretion pathway. Oxalobacter formigenes has furthermore been shown to promote active excretion of oxalate, possibly through interaction with SLC26 transporter proteins that enhance the oxalate flux from plasma to small bowel (Hatch et al., AJPGLP, 2011, 300 G461-G469; Hatch and Freel, Urolithiasis, 2013).

Compositions comprised of oxalate-reducing bacteria, such as Oxalobacter formigenes for use in methods for treating oxalate-related conditions have previously been disclosed in the art, such as in U.S. Pat. Nos. 6,200,562, 6,355,242, WO2007075447, and WO2005123114.

WO2017216165 A1 by the same applicant discloses improved pharmaceutical compositions comprising oxalate-degrading bacteria, Oxalobacter formigenes shown to be effective in the treatment of oxalate-related disorders. The pharmaceutical compositions were proven to be particularly useful for their purpose by using specific excipients and high doses of Oxalobacter formigenes having a high oxalate-reducing activity. The entire content of said publication is hereby incorporated herein by reference.

### SUMMARY OF THE INVENTION

Systemic oxalosis with cardiac involvement is a condition characterised by a decrease in the heart muscle function, resulting in a loss of the ability of the heart to contract. To overcome or at least mitigate the problems still remaining in the art and recited herein, there is provided herein a method for the use in the treatment or prevention of systemic oxalosis with cardiac involvement in a subject, said method comprising administering to said patient a pharmaceutical composition comprising viable dried Oxalobacter formigenes, said pharmaceutical composition being present in an enteric-coated capsule and wherein the oxalate-degrading activity in vitro of said Oxalobacter formigenes present in said pharmaceutical composition is no less than (NLT) 100 mmol oxalate/capsule/19 hours.

As previously disclosed in WO2017216165 the pharmaceutical composition for use herein has an optimized composition of excipients in combination with a hundred-fold higher concentration of viable Oxalobacter formigenes cells than in previous formulations, said composition possessing a high, preferred, oxalate-degrading activity.

Surprisingly and unforeseen, the oral administration of such a pharmaceutical composition to a subject suffering from systemic oxalosis with cardiac involvement rendered it possible to achieve a beneficial effect on the heart muscle function in PH patients on dialysis in addition to a stabilising or reducing effect on plasma oxalate levels. This is the first time ever that a stabilising or even an improved effect on cardiac function parameters, implicating a reversed effect on the calcification of the heart muscle, has been shown in patients suffering from systemic oxalosis with cardiac involvement.

Despite the previous knowledge within this field, it was unexpected that administration of the pharmaceutical composition as described herein could reverse the progression of cardiac involvement towards restoring the contracting function by reducing the stiffness of the heart. Previously, it was believed that such damages to the function of the heart were permanent and thereby irreversible.

The herein presented use of a pharmaceutical composition and associated method provides a novel and improved way to treat patients on dialysis suffering from systemic oxalosis with cardiac involvement. Examples of such patients are subjects in need of a kidney transplant, or subjects previously having been through one or several kidney transplantations.

### BRIEF DESCRIPTION OF THE FIGURES

FIG. 1a) shows a graph illustrating that the mean cardiac function was improved across the study population during the study period up to 52 weeks. Results show mean values (SD) for Traditional Echocardiography (LVEF (Left Ventricular Ejection Fraction), %) at baseline (n=6), week 24 (n=5) and week 52 (n=6). The normal range for LVEF is 55-65%. FIG. 1a) reflects the patients that had completed the study at the time. At the time of baseline shown in FIG. 1b), more patients had been able to join the study.

FIG. 1b) illustrates an improvement of the cardiac function across the study population during the study period up to 104 weeks. FIG. 1b) involves all 8 patients that started the study with LVEF measuring stated in the study. Some patients dropped out during the study. N=5 patients completed 24 months of treatment. The conclusions from the study are not affected by the study dropouts.

FIGS. 2 a) through f) show scatter plots of individual values of Global Longitudinal Heart Strain, GLS (%), in relation to total plasma oxalate (μmol/L) in a study population. The normal range for GLS is <−18%.

FIG. 3a) shows a graph of the primary endpoint of a clinically relevant reduction in total plasma oxalate levels of the study population. The p-value is based on the MMRM (mixed model of repeated measures) statistical methodology including all values over time from baseline to week 52.

FIG. 3b) shows a graph of the primary endpoint of a clinically relevant reduction in total plasma oxalate levels of the study population. The p-value is based on the MMRM (mixed model of repeated measures) statistical methodology including all values over time from baseline to week 104.

FIG. 3c) shows a graph of the primary endpoint of a clinically relevant reduction in free plasma oxalate levels of the study population. The p-value is based on the MMRM (mixed model of repeated measures) statistical methodology including all values over time from baseline to week 104.

FIGS. 4a) and b) show graphs over mean (SD) GLS plotted over mean (SD) total and free plasma oxalate, respectively.

FIGS. 5a) and b) show graphs over mean (SD) LVEF plotted over mean (SD) total and free plasma oxalate, respectively.

### DETAILED DESCRIPTION OF THE INVENTION

### Definitions

All words and terms used herein shall be considered to have the same meaning usually given to them by the person skilled in the art, unless another meaning is apparent from the context.

“Systemic oxalosis” is a condition involving deposition of oxalate in various extra-renal tissues leading to a systemic involvement of oxalate. Such tissues can be e.g. the bone, soft tissue, heart, nerves, joints, skin, retina and other visceral lesions.

“Systemic oxalosis with cardiac involvement” as referred to herein, is a condition characterized by a decrease in the function of the heart muscle due to reduced mechanical strain decreasing the contractibility of the heart. The reduced mechanical strain can result from a build-up of calcium oxalate deposits in the heart tissue, restricting the mechanical ability of the heart muscle to contract and accurately transport arterial blood. Herein, this condition may be identified in a subject by said subject having an impaired LVEF or GLS at the initiation of treatment, or when untreated, as further defined herein. Herein, it is intended to treat or prevent systemic oxalosis with cardiac involvement by facilitating a restoration and/or improvement of the contracting strain of the heart in said subject. This means that the treatment or prevention makes it possible to regain some of the original mechanical functions of the heart muscle. This was previously not believed to be possible. Instead, treatments have aimed at stopping or reducing the progression of the disease once it has reached a certain phase. This is the first time it has been shown an effect improving the mechanical function of the heart muscle.

Such a restoration and/or improvement involves reducing a stiffness in the heart muscle wall that has been built up by the condition. In turn, reducing a stiffness in the heart muscle wall will provide for a restoration of a contracting strain of the heart muscle. A treatment or prevention of systemic oxalosis with cardiac involvement is also intended to stabilize or reduce plasma oxalate levels in said subjects while at the same time restoring the contracting function.

“Ejection Fraction” or “Left Ventricular Ejection Fraction” (abbreviated LVEF) as referred to herein is the volumetric fraction of blood ejected from the left ventricular chamber, with each contraction of the heart (or heartbeat). Even if only “Ejection Fraction” is referred to, it generally and also herein refers to the left ventricular ejection fraction. EF or LVEF is widely used for measuring pumping efficacy of the heart and for indicating the severity of heart failure. LVEF is most commonly assessed by traditional echocardiographic methodologies. Krishnasamy et al, 2015, PLOS ONE, 10(5)).

“Global Longitudinal Strain” (GLS), referred to herein, is a technique for detecting and quantifying subtle disturbances in left ventricular (LV) systolic function. GLS reflects the longitudinal contraction of the myocardium. GLS is usually assessed using automated speckle-tracking echocardiography (Krishnasamy et al, 2015, PLOS ONE, 10(5)).

“Oxalate-degrading activity in vitro” is the oxalate metabolizing capacity of O. formigenes as measured by the amount of formate generated from replication and/or oxalate degradation during culture of cells in oxalate containing medium. Stoichiometrically, one mole of formate is generated for each mole of oxalate degraded or consumed (Stewart et al., FEMS Microbiology Letters 230, 2004, 1-7).

“Glomerular Filtration Rate” (GFR) is the flow rate of filtered fluid in ml/min., body surface area through the glomeruli of the kidney, which is a key indicator of renal function.

“Estimated Glomerular Filtration Rate (eGFR)” is an estimate of the Glomerular Filtration Rate, and it is based on a patient's serum creatinine level combined with several other factors. Different equations are used for adults and children. The equation includes the serum creatinine concentration and some or all of the following parameters; age, ethnicity, gender, height, weight (depending on equation type), blood urea nitrogen (BUN) and cystatin C. The commonly used equations include Cockraft and Gault (1976), Modification of Diet in Renal Disease (MDRD) (1999) and Chronic Kidney Disease Epidemiology Collaboration (CKD-EPI) (2009) for adults and Schwarz (2009) for children.

A “primary endpoint” is the main predefined parameter that is measured in a study to determine efficacy of a given treatment (e.g., effect difference between the treatment group and the control group, or the difference from baseline within the treatment group itself).

The term “dry weight” as referred to herein, is intended to mean the weight of a composition wherein most of the water has been removed therefrom, such as by a drying process (e.g. lyophilisation).

The terms “cryopreserving agents” and “excipients” may sometimes be used interchangeably herein. However, the term “cryopreserving agent” is herein intended to refer to an agent used to preserve cell viability when cooling to sub-zero centigrade temperatures. Cryopreservation is a process that is well-known in the art. Herein, the compositions also comprise one or more “excipients”, which term is mainly used to describe other ingredients present in the composition, such as ingredients added thereto in order to, in other manners than cryopreservation, preserve stability or prevent degradation of the composition, as well as to absorb moisture. A purpose of an excipient can also be to achieve desired powder properties (e.g. free flowing powder).

An “enteric-coated” capsule as defined herein, refers to a capsule having outer surface coating characteristics, which makes it suitable for targeted delivery of a pharmaceutical agent, present therein, to a specific segment of the intestine. An enteric coating can also be described as a barrier applied on an oral drug preventing it from dissolution or disintegration in the gastro-intestinal environment. Accordingly, such a coating allows the drug to survive the acidic and enzymatic environment of the stomach and the duodenum. Herein, the term “capsule” may have any suitable form as long as it is encapsulating the pharmaceutical composition in a manner, which makes it suitable for transport and administration to the small intestine, such as the ileum, of a subject after the oral administration.

Herein the term “subject” may be used interchangeably with the terms individual and patient.

Compositions “comprising” one or more recited elements may also include other elements not specifically recited.

The singular “a” and “an” shall be construed as including also the plural.

### DETAILED DESCRIPTION

There is provided herein a pharmaceutical composition comprising viable dried Oxalobacter formigenes for use in a method for the treatment or prevention of systemic oxalosis with cardiac involvement in a subject, wherein said pharmaceutical composition comprises at least 10⁹ CFUs, such as up to 10¹² CFUs, of viable dried Oxalobacter formigenes present in an enteric-coated capsule and wherein the oxalate-degrading activity in vitro of said Oxalobacter formigenes is no less than 100 mmol oxalate/capsule/19 hours.

Equally, there is also provided herein a method for the treatment and/or prevention of systemic oxalosis with cardiac involvement in a subject, said method comprising administering a pharmaceutically effective amount of a pharmaceutical composition comprising at least 10⁹ CFUs, such as up to 10¹² CFUs, of viable dried Oxalobacter formigenes present in an enteric-coated capsule, wherein the oxalate-degrading activity in vitro of said Oxalobacter formigenes is no less than 100 mmol oxalate/capsule/19 hours, to a subject in need thereof.

Equally, there is also provided herein the use of a pharmaceutical composition comprising viable dried Oxalobacter formigenes in the manufacture of a medicament for the treatment or prevention of systemic oxalosis with cardiac involvement in a subject, wherein said pharmaceutical composition comprises at least 10⁹ CFUs, such as up to 10¹² CFUs, of viable dried Oxalobacter formigenes present in an enteric-coated capsule and wherein the oxalate-degrading activity in vitro of said Oxalobacter formigenes is no less than 100 mmol oxalate/capsule/19 hours.

The treatment with a pharmaceutical composition as presented herein is intended to proceed continuously for a period of months or years, i.e. to constitute a long-term treatment, during which period said subject may also undergo dialysis as described elsewhere herein.

A subject suffering from systemic oxalosis with cardiac involvement may be characterized herein by having a Left Ventricular Ejection Fraction (LVEF) of 55%, such as 50%, or even ≤40%. Said subject may also alternatively or additionally be characterized by having a Global Longitudinal Strain (GLS) of >−18%, such as >−15%, or even >−10%. Such a value is usually present when treatment is initiated and/or in the beginning of the treatment with a pharmaceutical composition as defined herein.

Occasionally, such as when a long-term treatment with the pharmaceutical composition has been initiated, the GLS value and/or the LVEF value in said subject may temporarily vary in both directions, i.e. the value(s) may improve or worsen in a fluctuating pattern. In addition, the values can vary independently of each other, i.e. one could improve while the other could worsen.

However, over time, such as after a period of months or years of continuous treatment with the pharmaceutical composition, it is intended that said subject will stabilise at (a) healthy or improved GLS and/or LVEF value(s). Healthy GLS and/or LVEF value(s) may correspond to values such as at −20% (±1-3%, or rather not higher than −18) such as at −18 or lower −18, such as −20 or −25 (GLS)) or 55%, such as about 55 to 70%, respectively (LVF) in a healthy subject (Lagies et al., Echocardiography, 2014, DOI:10.1111/echo.12842, Lagies, Circ. Heart failure. 2013; 6:e45-e47).

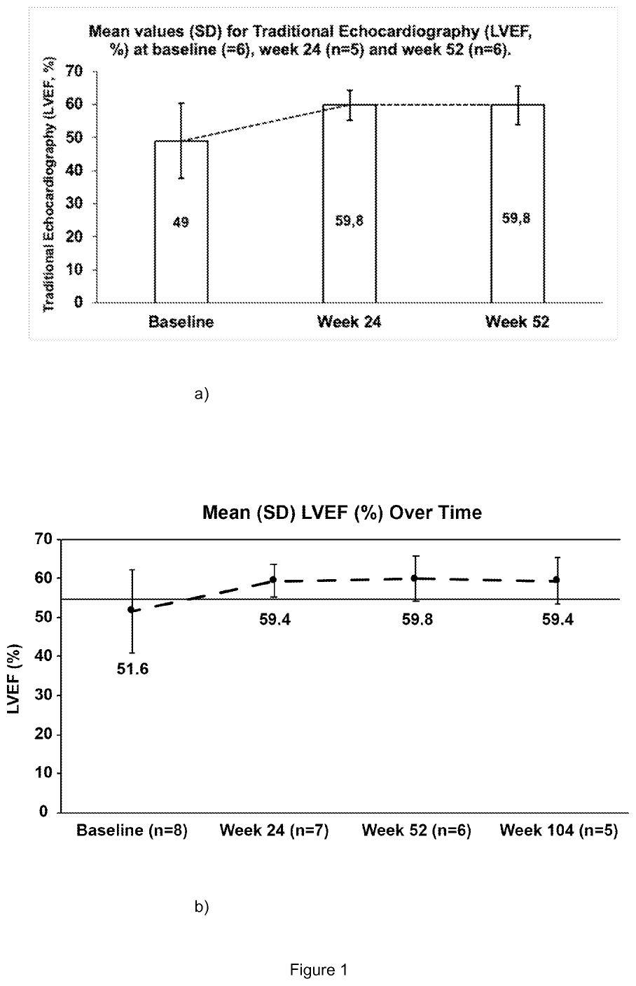

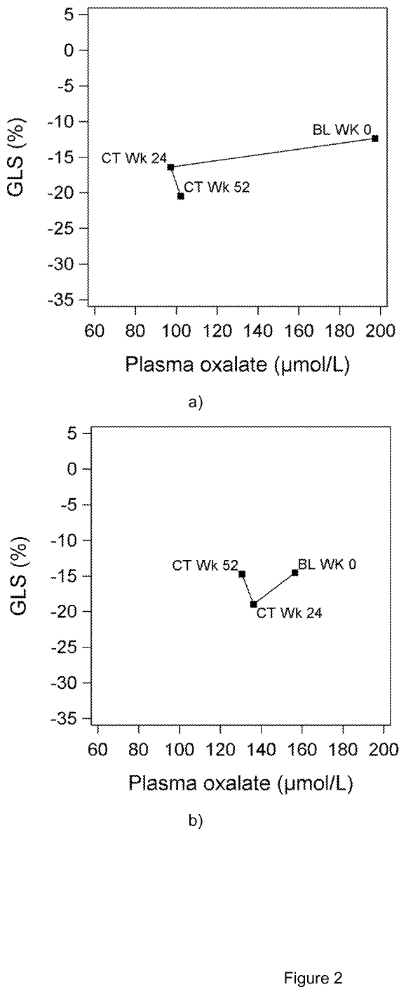

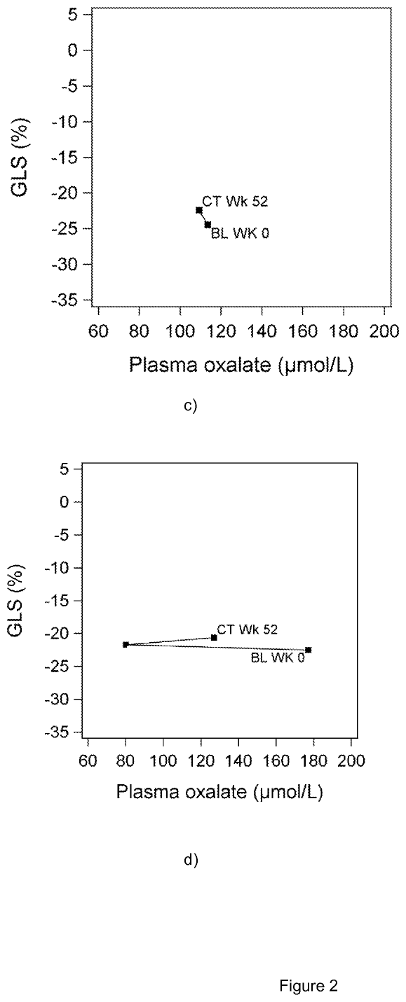

As shown in FIGS. 1a) and 1b), the cardiac function was improved across the study population during the study period. Notably, the LVEF is improved in four out of six patients in the study (52 weeks study). The two patients, who did not improve, were young patients with a normal heart function at baseline. Furthermore, in FIGS. 2 a) through f), where GLS is plotted against plasma oxalate over time for each individual patient, it can be seen that patients start at week 0 with elevated plasma oxalate. GLS is for four patients impaired at levels >−18%. Notably, the mean of all patients, plasma oxalate reduced and GLS moved to lower values (i.e. the strain improved). Accordingly, this proves the successful use of a pharmaceutical composition presented herein in a method for the treatment or prevention of systemic oxalosis with cardiac involvement.

A subject treated with the pharmaceutical composition presented herein may be a subject who is on dialysis when the treatment is initiated. Said subject may be on a stable dialysis regimen throughout the treatment and has usually, in addition thereto, been on dialysis for at least four months before the treatment is initiated (optional).

Herein, said subject may be on a dialysis treatment as a consequence of an oxalate imbalance in the body of said subject.

Herein, a treatment or prevention of systemic oxalosis with cardiac involvement in a subject may comprise facilitating an improvement in and/or a restoration of a contracting function in the heart muscle of said subject. Said improvement in and/or restoration of a contracting function in the heart muscle may be characterized or defined by an improvement in a LVEF and/or a GLS value in said subject, as previously defined herein, such as when said GLS value in said subject is ≤−18 and/or said LVEF value in said subject is ≥55.

A subject mentioned herein may be suffering from end-stage renal disease (ESRD). Said subject may also be suffering from a Chronic Kidney Disease (CKD) of Stage 4 or 5 with risk for heart failure and/or impaired heart elasticity. The impaired heart elasticity is mainly a result of calcium oxalate deposits forming in the heart tissue resulting in a decrease in the heart contractibility. As previously mentioned herein, CKD stages are determined based on eGFR (using different equations also referred to herein). It is notable that not all patients within a certain CKD stage suffer from heart problems. Accordingly, a subject suffering from systemic oxalosis with cardiac involvement as defined herein may be suffering from CKD stage 4 or 5, or other stages. It may also not necessarily be connected with CKD. Accordingly, it should be considered a different subpopulation sometimes residing within the definition of a CKD of various stages. Such a subpopulation does not necessarily need to be on dialysis, but is likely to be.

Herein, a subject who is in need of treatment or prevention of systemic oxalosis with cardiac involvement has an estimated Glomerular Filtration Rate (eGFR) within the range of 0≤eGFR 20 ml/min, such as 0≤eGFR≤15 ml/min, 0≤eGFR 10 ml/min, 0≤eGFR 5 ml/min, or 0≤eGFR≤0.5 ml/min, at the initiation start of said treatment. This means that said subject has a severe decline in kidney filtering function. Herein, another relevant condition may therefore be that said subject has had, or will be in need of, organ transplantation, such as kidney transplantation.

The Pharmaceutical Composition

There is furthermore provided herein, a pharmaceutical composition for use in a method as defined elsewhere herein, wherein said pharmaceutical composition more specifically comprises:

(i) about 10% to about 25% by dry weight of Oxalobacter formigenes,

(ii) about 50% to about 65% by dry weight of sucrose; and

(iii) about 10% to about 30% by dry weight of one or more cryopreserving agents and/or excipients.

The pharmaceutical composition used according to the present disclosure comprises highly concentrated dried (e.g. lyophilized) bacteria of O. formigenes having a fast recovery time, a minimum viable cell count of Not Less Than (NLT) 10⁹ CFU/capsule (such as about 10⁹ to 10¹² CFU/capsule), and an oxalate degrading capacity of NLT of about 100 mmol oxalate/capsule/19 hours, such as about 200 mmol, about 300 mmol, about 400 mmol, about 500 mmol, or even up to about 2 mol oxalate/capsule/19 hours, or the like. Instead of per capsule it may also be referred to as per dose. This defines the activity of the bacteria selected for the preparation of the pharmaceutical composition, i.e. it allows disregarding certain batches of cells, which may contain less active cells when preparing the composition. The identification of bacteria possessing such an oxalate degrading activity may be performed in an assay measuring oxalate degrading activity as illustrated below.

Oxalate-Degrading Activity

Testing of the potency, i.e. the oxalate degrading activity of O. formigenes, is performed indirectly by measuring the amount of formate generated from oxalate degradation activity during culture of cells in oxalate containing media (60 mM oxalate “OxB” medium, Allison et al., 1985, Medium B). Samples are withdrawn and filtered, after incubation at 37° C. The concentration of formate is determined by High Performance Liquid Chromatography (HPLC) against a formate standard curve using a cation exchange column. Stoichiometrically, one mole of formate is generated for each mole of oxalate consumed (Stewart et al., 2004):

Oxalate→oxalyl-CoA→formyl-CoA+CO₂→formate

The assay for the present formulation measures accumulated oxalate degradation at about 19 hours, a time point where linearity between sample dilutions is observed and the cells have reached exponential phase.

By this route, the assay is allowed to discriminate active oxalate degrading activity from background metabolic activity.

Disintegration of Enteric-Coated Capsule

The structural characteristics of the enteric-coated capsule used may be described in a functional manner by e.g. referring to its ability to withstand disintegration in in vitro conditions simulating conditions in the gastrointestinal part of the body. Herein, the capsule is described both with regard to its ability to withstand disintegration in the stomach environment, and with regard to its ability to withstand disintegration for a limited period of time also in an intestinal environment. Hence, herein, the characteristics of the enteric-coated capsule are mainly described in relation to its ability to withstand disintegration during incubation in “Simulated Gastric Fluid” (SGF) and in “Simulated Intestinal Fluid” (SIF).

“Simulated Gastric Fluid” (SGF) is an artificial dissolution medium that is intended to represent stomach acid. It may prepared by dissolving sodium chloride and subsequently adding purified pepsin (e.g. derived from porcine stomach mucosa, with an activity of about 800 to 2500 units per mg of protein), in hydrochloric acid. The test solution has a pH of about 1.2±0.1. The temperature of the SGF is kept at about 37° C. and the concentration of the enzyme in the fluid is about 3.2 mg/ml.

“Simulated Intestinal Fluid” (SIF) is an artificial dissolution medium that is intended to represent intestinal fluid. It may prepared by dissolving potassium phosphate in water and adding sodium hydroxide and adjusting the pH to pH 6.8±0.1 and subsequently adding purified pancreatin. The temperature of the SIF is kept at about 37° C. and the concentration of the enzyme in the fluid is about 10 mg/ml.

Six capsules may be tested at the same time. Complete disintegration is defined as that state in which any residue of the unit, except fragments of insoluble coating or capsule shell, remain on the screen of the test apparatus or adhere to the lower surface of the discs, is a soft mass having no palpably firm core. The acceptance criterion for SGF is met if all six capsules show no evidence of disintegration or rupture permitting the escape of contents. The acceptance criteria for SIF are met if all six capsules show evidence of a start of disintegration within 60 minutes. A procedure for disintegration of tablets and capsules is also described in the European Pharmacopoeia 5.0, 2.9.1 (Test A).

In accordance therewith, the enteric-coated capsule can be defined as showing essentially no disintegration within one hour of incubation in Simulated Gastric Fluid (SGF) having a pH of about 1.2±0.1 and comprising about 3.2 mg/ml of pepsin at a temperature of about 37° C., but wherein a start of disintegration of said capsule is detected within about one hour in Simulated Intestinal Fluid (SIF) having a pH of about 6.8±0.1 and comprising about 10 mg/ml of pancreatin at about 37° C. These characteristics of the enteric-coated capsule explain that it will survive the acidic environment in the stomach, and it will also last for some time in the intestinal environment, thereby efficiently targeting delivery of Oxalobacter to the small intestine and/or to the ileum.

Notably, a capsule herein, having the characteristics of a start of disintegration within about one hour in Simulated Intestinal Fluid (SIF) having a pH of about 6.8±0.1 and comprising about 10 mg/ml of pancreatin at about 37° C., has previously been proven particularly useful. Hence, this means that the capsule remains for quite some time in the intestinal tract, resulting in a slow release of the pharmaceutical composition. This is a difference compared to other capsules, e.g. where a complete disintegration (or collapse) is seen within about one hour in Simulated Intestinal Fluid (SIF) having a pH of about 6.8±0.1 and comprising about 10 mg/ml of pancreatin at about 37° C. As previously mentioned herein, the capsule may be a gelatin capsule, such as a hard gelatin capsule, or another similar capsule providing similar characteristics, thus resulting in a preferred release profile of the drug.

Examples of polymer coatings that may be used to prepare a coating for a capsule comprising a pharmaceutical composition according to the present disclosure that withstands disintegration within the above defined limits are e.g. methacrylic acid polymers including methacrylic acid copolymers and anionic methacrylic acid copolymers such as provided by the coatings of Eudragit®. These may e.g. be purchased from Evonik Industries (http://eudragit.evonik.com) and may also be prepared by a person skilled in the art, further optionally taking into account additional available information available to the skilled person, such as Remington's Pharmaceutical Sciences. The selection of the appropriate polymers to produce or coat a capsule may be performed by the skilled person by taking into account the particulars presented herein.

The capsule may be a gelatin capsule, such as a hard gelatin capsule, or another similar capsule providing similar characteristics.

Naturally, the compositions described herein may comprise some water, such as about 3% water.

A pharmaceutical composition may also consist of the above described ingredients and optionally water.

The contents of the pharmaceutical compositions mentioned herein are described in percentages and in dry weight. This dry composition has been obtained through a drying-process, such as freeze-drying, and may in such a context also be referred to as a powder composition or a lyophilized/freeze-dried (powder) composition. When drying a composition, there may be some water left which is illustrated by the compositions comprising a certain amount of water.

More specifically, there is provided a pharmaceutical composition comprising about 10% to about 20%, about 10% to about 25%, about 15% to about 20%, about 15% to about 25%, about 17% to about 21%, about 18% to about 22%, or about 18% to about 20%, such as about 15%, 16%, 17%, 18%, 19%, 20%, 21% or 22% by dry weight of Oxalobacter formigenes in said composition.

Furthermore, a pharmaceutical composition herein may comprise about 50% to about 65% by dry weight of sucrose, such as about 52% to about 62%, about 54% to about 60%, or about 56% to about 58%, such as about 54%, 55%, 56%, 57%, 58%, 59%, 60%, 61%, 62%, 63%, 64% or 65%.

The amount(s) of one or more cryopreserving agents and/or excipients may be about 10% to about 30% by dry weight, such as about 15%, 16%, 17%, 18%, 19%, 20%, 21%, 22%, 23%, 24%, 25%, 26%, 27%, 28%, 29% or 30% of one or more cryopreserving agents and/or excipients.

Notably, all % amounts mentioned herein may generally at least vary about 1-3%, i.e. ±1-3% depending on the exact manufacturing process and cell density used for preparing the pharmaceutical composition. The compositions are prepared from a cell paste and excipients in solution and then frozen and lyophilized by procedures known in the art.

In a pharmaceutical composition presented herein, the excipients and/or cryopreserving agents may be selected from the group consisting of maltodextrin, oligofructose and alginate. Other, equally functional and structurally similar agents may also be used.

In a pharmaceutical composition as disclosed herein, about 15% to about 21%, such as about 16% to about 19%, such as about 15%, 16%, 17%, 18% or 19% by dry weight of maltodextrin may be used.

There is further provided herein a pharmaceutical composition comprising:

i) about 1% to about 5% by dry weight of oligofructose; and

ii) about 0.5% to about 2% by dry weight of alginate. Said pharmaceutical composition may also comprise water, such as in the amount of about 1% to about 5% by weight of said composition.

A pharmaceutical composition as presented herein may also comprise about 0.5% to about 1.5% by dry weight of alginate, or an agent similar to alginate, such as about 1%. Oligofructose may be about 1%, 2%, 3%, 4% or 5% by dry weight.

There is further provided herein a pharmaceutical composition comprising:

about 17% to about 22% by dry weight of Oxalobacter formigenes,

about 52% to about 62% by dry weight of sucrose;

about 17% to about 25% by dry weight of one or more cryopreserving agents and/or excipients.

Furthermore, there is provided a pharmaceutical composition comprising:

about 19% by dry weight of Oxalobacter formigenes,

about 57% by dry weight of sucrose;

about 21% by dry weight of one or more cryopreserving agents and/or excipients, and

remaining water. The cryopreserving agents and/or excipients may be alginate, maltodextrin and/or oligofructose, and may be present in the amounts of about 1% alginate, about 17% maltodextrin and about 3% oligofructose by dry weight.

The pharmaceutical composition or the enteric-coated capsule defined herein may be administered in amounts containing about 10⁹ to about 10¹² CFUs of O. formigenes at least twice a day for a continuous period of time, such as a period lasting for at least months or years, to a subject in need thereof. Such a period may last from 1, 2, 3, 4, 5, 6 or up to 12 months, 1, 2, 3, 4, or 5 or even more years.

Said subject mentioned herein may be a mammal, such as a human.

O. formigenes can be of genotype 1 or genotype 2; both types are naturally occurring. The pharmaceutical compositions for use according to the present disclosure preferably comprise O. formigenes genotype 1.

As previously mentioned herein, all aspects of the disclosure are equally applicable to a method of treatment of a condition as defined herein or to the use of said pharmaceutical composition in the manufacture of a medicament in the treatment or prevention of a condition as defined herein.

The present disclosure will now be illustrated by the following experimental section without intending to be limited thereto as it merely illustrates different ways of performing the invention.

### EXPERIMENTAL SECTION

Drug

The drug was supplied as enteric-coated capsules.

The study drug is supplied as enteric-coated, size 4, gelatin capsules. One capsule contains ≥10⁹ colony forming units (CFU). Details on the product are described in Table 1.

TABLE 1
Details of the drug

| Parameter | High dose |
|---|---|
| Active Substance | Lyophilised O. formigenes, strain HC-1 |
| Route of Administration | Oral |
| Dose Form | Enteric-coated capsule |
| Viable Cell Count | ≥10⁹ CFU/dose |
| Excipients | Oligofructose, Maltodextrin, Alginate, |
| | Sucrose, Microcrystalline Cellulose |

The optimal (in vitro) oxalate degrading capacity of the capsules is MOO mmol oxalate/capsule/19 h. This dose and enzyme activity is needed to ensure delivery of sufficient amount of viable O. formigenes to the relevant part of the gastrointestinal tract, as the bacteria need to survive transit through the stomach and upper small intestine and withstand the dilution effect from the normal gut microbiota. It is a competitive environment particularly given that O. formigenes are anaerobic and utilise only oxalate as an energy source.

Treatment Schedule 52 Week Study

Patients on a stable dialysis regimen suffering from systemic oxalosis with cardiac involvement had a LVEF of ≤55% when the treatment was initiated.

The study involved two parts: the first 14 weeks included baseline, treatment and follow-up and the second part included 52 weeks of continued and uninterrupted treatment.

The first 14 weeks of the study involved:
Patients were monitored for 4 weeks prior to initiation of treatment with Oxalobacter formigenes study medication to collect baseline (reference) data.
Patients were treated for 6 weeks with the pharmaceutical composition twice daily.
Patients were then evaluated for safety and clinical status evaluation during a 4 week period before continued treatment.

The continued 52 weeks of treatment (“Year 1) involved:
Uninterrupted treatment for all patients during 52 weeks continuous weeks (“Year 1”) with study medication
Safety and clinical data evaluation.

The dose of the pharmaceutical composition that was administered to the subjects of the study was one capsule for oral administration with water twice daily with breakfast and dinner. One capsule corresponds to one dose: ≥10⁹ CFU/dose of dried viable Oxalobacter formigenes.

Patients were on a stable haemodialysis (HD), only peritoneal dialysis (PD) or combined HD and PD regimen. The choice of dialysis regimen was decided upon by the investigator and individualized as appropriate to control plasma oxalate concentration by dialysis. The PD regimen (e.g. number of days/week, hours per treatment, volume of the dialysis buffer etc.) and HD regimen (e.g. number of days/week; hours per treatment; blood and dialysate flow rates; and HD membrane etc.) were then maintained constant for the duration of the study.

The morning pre-dialysis session total plasma oxalate concentration was measured at weeks 2, 4, 6, 8, 10, 12 and 14 during the first 14 weeks of the study, and then monthly throughout Year 1 in the continued treatment period.

Speckle Tracking Echocardiography (STE) and traditional echocardiography were used to assess changes in the LV contractibility. STE and traditional echocardiography were performed at patient baseline screening (alternatively before start of continued treatment) and repeated every 6 months during the continued treatment period. The examination was performed locally using specific equipment. Images were interpreted by a central reader who was blinded to examination date and patient identity and other clinical assessments, including plasma oxalate concentrations. STE results were evaluated for changes in all possible parameters and in particular for changes in Global Longitudinal Strain. Traditional echocardiographic parameters were also evaluated including Left Ventricular Ejection Fraction.

A positive treatment effect would be an improved myocardial function based on the above-mentioned parameters.

All patients that completed the 52 week study were allowed to proceed for another 52 weeks (104 weeks study). The dialysis regime and the sample schedule were kept the same during the continuation of the study.

Patients

The overall purpose of the study was to assess whether the pharmaceutical composition was capable of improving cardiac involvement due to systemic oxalosis in patients with CKD stage 4 or 5, while stabilising or reducing the plasma oxalate concentration, and maintaining an acceptable benefit/risk balance. PH patients in ESRD on dialysis were selected for the study as they will potentially benefit most from treatment, having a more severe form of the disease and typically show higher plasma oxalate levels and more oxalate deposits than patients without PH. Patients with severe PH and on progression to ESRD (chronic kidney disease (CKD) stage 5) can also be on dialysis with a preventive purpose while not yet being anuric (i.e. still being able to produce urine). In addition to high plasma oxalate levels and the high burden of oxalate deposits, these patients also show very high urinary oxalate levels.

All patients in this study reported End Stage Renal Disease, secondary hyperparathyroidism and (nephrogenic) anaemia. Other conditions concerned nephrocalcinosis, nephrolithiasis, oxalosis, hypertension, erosive gastritis and renal transplant failure.

The total plasma oxalate concentrations in the patients over time, before starting the treatment, varied between about 100 to 200 μmol/L.

Results

Cardiac Involvement: Cardiac Function Improved Across Study Population

As illustrated in FIGS. 1a) and 1b), mean values for traditional echocardiography (LVEF, %) improved across the study population during the 2 year study period. The improvements in LVEF are indicative of improved cardiac involvement from oxalate deposit-induced ventricular strain, i.e. indicating that systemic oxalosis is decreasing with the treatment. As an example, patients with LVEF ≤40% at baseline experienced a normalization in LVEF as plasma oxalate reduced. As illustrated in FIGS. 2a) through to f) where GLS is plotted against plasma oxalate over time for each patient, it can be seen that patients start at week 0 with elevated plasma oxalate. GLS is for four patients impaired at levels >−18%. In all patients, plasma oxalate reduced and GLS moved to lower values, i.e. individual graphs move from the right side of the diagram towards lower left side of the diagram.

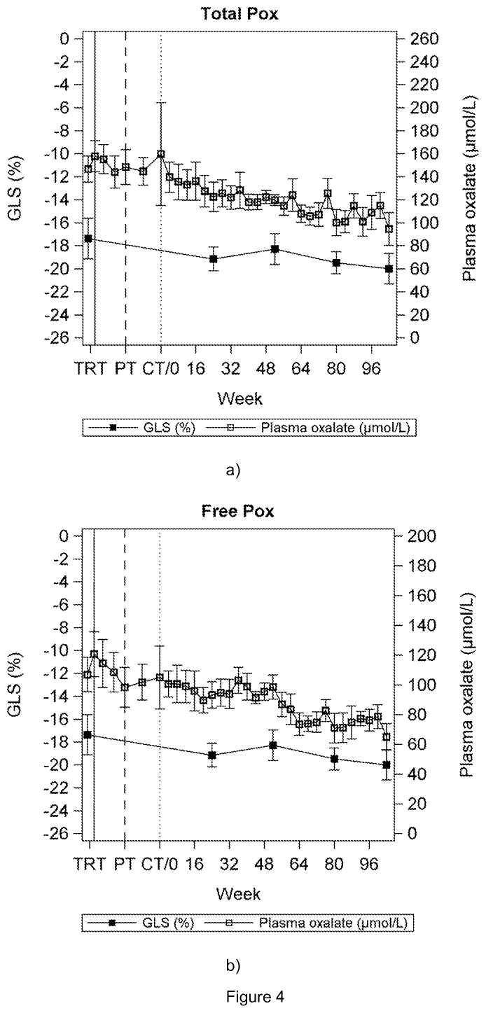

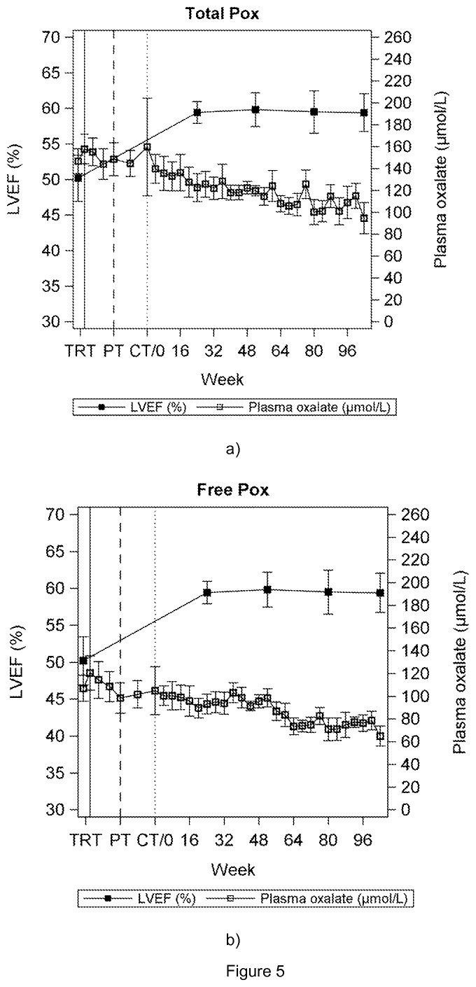

In both FIGS. 4 and 5, it is illustrated that the ability of the heart to contract is improved over time as the total and free plasma oxalate concentrations decrease. The heart function is improved as the plasma oxalate levels goes down. The fact that total and free plasma oxalate is getting closer to each other is an indication that the deposits of oxalate are dissolving.

Surprisingly, no patient had a deterioration in cardiac function during a two year treatment with the pharmaceutical composition according to the invention.

Plasma Oxalate Burden: Clinically Relevant Reduction in Total Plasma Oxalate

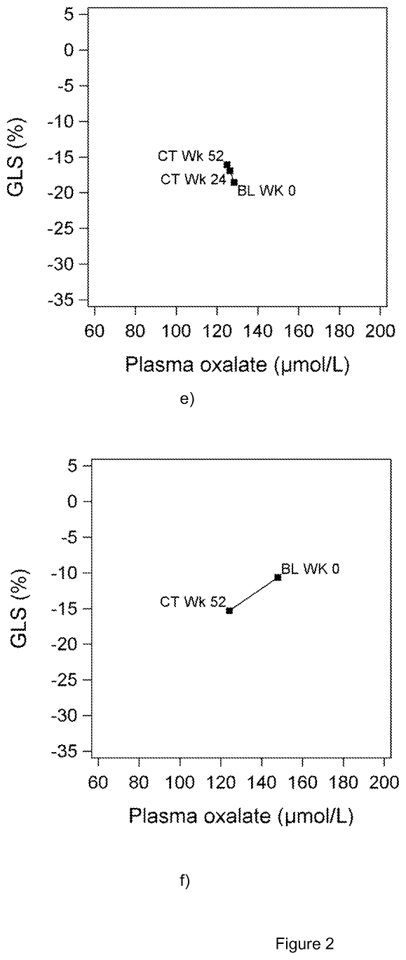

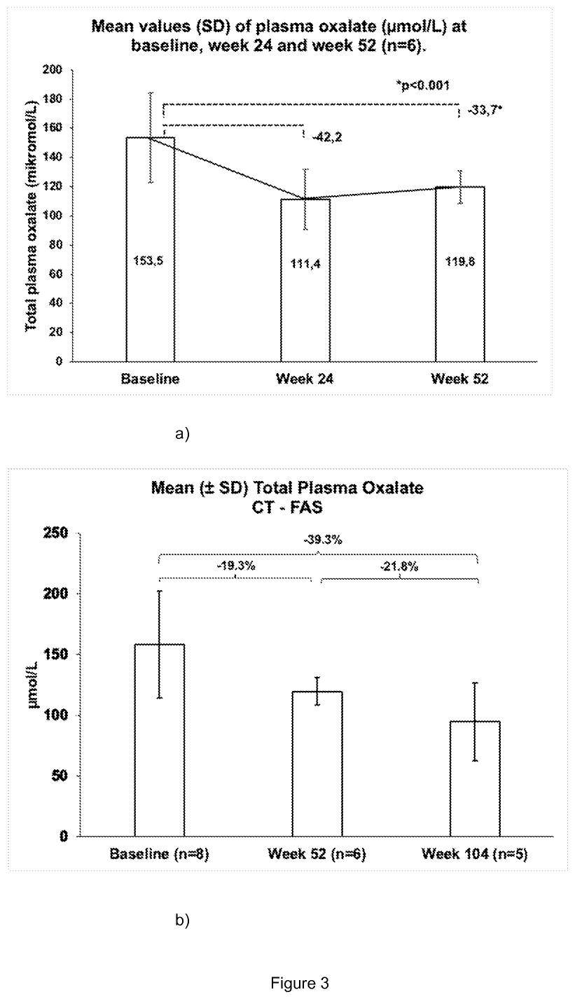

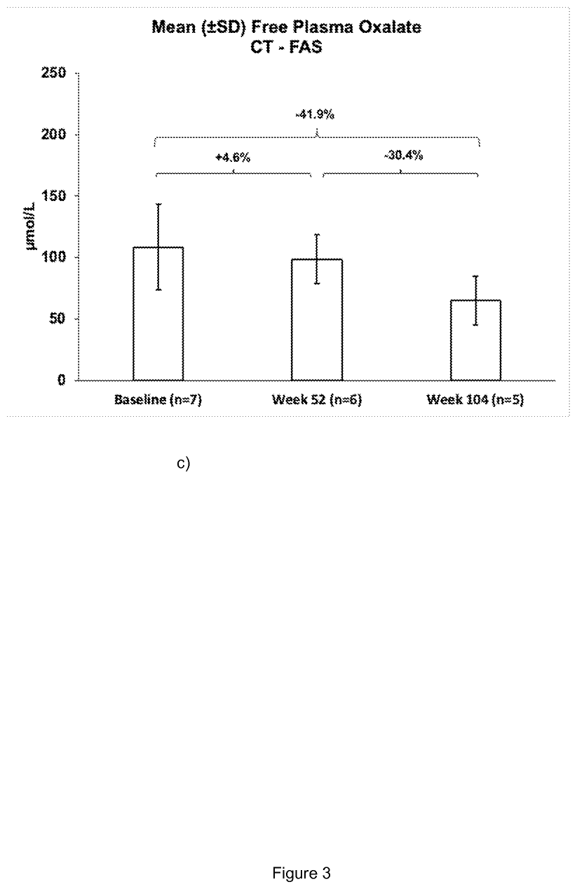

Given the natural history data of Pox (Plasma oxalate) in PH and non-PH patients with ESRD, it would be expected to observe an increase in Pox levels over time in patients in progression towards, or in ESRD. However, as shown in FIG. 3 (FIGS. 3a) and 3b)), total and free plasma oxalate concentrations decreased as a result of treatment with Oxalobacter formigenes.

Treatment with the pharmaceutical composition for up to 52 weeks was associated with a reduction and a stabilisation of total plasma oxalate concentrations. Accordingly, data supports that treatment over 52 weeks improves the clinical situation of patients with primary hyperoxaluria on dialysis. Mean total plasma oxalate was significantly lower at treatment week 52 compared to baseline (n=6): 119.8 micromol/L at treatment week 52 compared to 153.5 micromol/L at baseline with a p-value <0.001 based on MMRM (mixed model of repeated measures) including all values over time from baseline to week 52.

Treatment with the pharmaceutical composition for up to 104 weeks was also associated with a reduction and a stabilisation of total plasma oxalate concentrations. Mean total plasma oxalate was significantly lower at treatment week 104 compared to baseline: 94.6 micromol/L at treatment week 104 compared to 158.3 micromol/L at baseline (n=8) with a p-value<0.001 based on MMRM (mixed model of repeated measures) including all values over time from baseline to week 104.

Similar changes were observed for free oxalate plasma concentrations, see FIG. 3a). Total plasma oxalate is the total oxalate found in plasma including oxalate crystals associated to protein. Free or soluble oxalate, as referred to herein, is the oxalate not bound to biomolecules or entities in blood or plasma. The decrease in total plasma oxalate concentrations shown in this study is clinically relevant and indicative of a reducing crystal burden in plasma, and a halted or slowed disease progression.

A kidney patient on dialysis without primary hyperoxaluria normally has a free plasma oxalate (Pox) concentration of around 50 μmol/L. The graph shows that free Pox was essentially normalized after 2 years treatment with the pharmaceutical composition. The difference between total and free oxalate is also decreasing with time. This observation indicates that oxalate is mobilized, i e that crystal-bound oxalate decreases as more and more oxalate is solubilized.

Summary of Results

In summary, the pharmaceutical composition described herein has the advantages of improving left ventricular function indicating that systemic oxalosis is decreasing. In addition, the treatment reduces the risk of transplant failure by lowering pre-transplant oxalate burden, avoids increases in dialysis frequency, and facilitates the transplantation procedure.

Our data at 104 weeks also show that the difference between total and free plasma oxalate is decreasing. As previously mentioned herein, this is an indication that the deposits of oxalate are dissolving and that a contracting function of the heart has been regained.

### REFERENCES

1. Cochat P. et al., N Engl J Med 369, 649-658, 2013
2. Hoppe B, Nature reviews Nephrology, 8, 467-475, 2012
3. Krishnasamy et al., Neprol Dial Transplant (2014) 29:1218-12254
4. Lagies et al. Echocardiography, 2015 August; 32(8), 1250-605 Costello et al., JASN, 1991 (1) 1289-1298
5. Hatch et al., AJPGLP, 2011, 300 G461-G469
6. Hatch and Freel, Urolithiasis, 2013
7. Krishnasamy et al, 2015, PLOS ONE, 10(5)
8. Stewart et al., FEMS Microbiology Letters 230, 2004, 1-7
9. Allison et al., 1985

1. A pharmaceutical composition comprising viable dried Oxalobacter formigenes for use in a method for the treatment or prevention of systemic oxalosis with cardiac involvement in a subject, wherein said pharmaceutical composition comprises at least 10⁹ CFUs, such as up to 10¹² CFUs, of viable dried Oxalobacter formigenes present in an enteric-coated capsule and wherein the oxalate-degrading activity in vitro of said Oxalobacter formigenes is no less than 100 mmol/capsule/19 hours.

2. The pharmaceutical composition for use according to claim 1, wherein said subject is characterized by having a Left Ventricular Ejection Fraction (LVEF) of about ≤55%.

3. The pharmaceutical composition for use according to claim 1 or 2, wherein said subject is characterized by having a Global Longitudinal Strain (GLS) of about ≥−18%.

4. The pharmaceutical composition for use according to any one of claims 1 to 3, wherein said subject is on dialysis or will be on dialysis once the treatment is initiated.

5. The pharmaceutical composition for use according to any one of claims 1 to 4, wherein said subject is on a stable dialysis regimen throughout the treatment, optionally wherein said subject has been on dialysis at least four months before the treatment is initiated.

6. The pharmaceutical composition for use according to any one of the preceding claims, wherein as a consequence of an oxalate imbalance in the body of said subject, said subject is on a dialysis treatment.

7. The pharmaceutical composition for use according to any one of the preceding claims, wherein the treatment or prevention of systemic oxalosis with cardiac involvement in a subject comprises facilitating an improvement in and/or a restoration of a contracting function in the heart muscle of said subject.

8. The pharmaceutical composition for use according to claim 7, wherein said improvement in and/or restoration of a contracting function in the heart muscle is characterized by an improvement in a LVEF and/or a GLS value in said subject, such as when said GLS value in said subject is ≤−18 and/or said LVEF value in said subject is ≥55.

9. The pharmaceutical composition for use according to any one of the preceding claims, wherein said subject is suffering from end-stage renal disease (ESRD).

10. The pharmaceutical composition for use according to claim 9, wherein said subject is suffering from a Chronic Kidney Disease (CKD) of Stage 5 with risk for heart failure and/or impaired heart elasticity.

11. The pharmaceutical composition for use according to any one of the preceding claims, wherein said subject has an estimated Glomerular Filtration Rate (eGFR) within the range of 0≤eGFR≤20 ml/min, such as 0≤eGFR≤15 ml/min, 0≤eGFR≤10 ml/min, 0≤eGFR≤5 ml/min, or 0≤eGFR≤0.5 ml/min, when said treatment is initiated.

12. The pharmaceutical composition for use according to any one of the preceding claims, wherein said subject has had, or will undergo, organ transplantation, such as kidney transplantation.

13. The pharmaceutical composition for use according to any one of the preceding claims, wherein said pharmaceutical composition comprises:
(i) about 10% to about 25% by dry weight of Oxalobacter formigenes,
(ii) about 50% to about 65% by dry weight of sucrose; and
(iii) about 10% to about 30% by dry weight of one or more cryopreserving agents and/or excipients.

14. The pharmaceutical composition for use according to any one of the preceding claims, wherein said composition comprises:
(i) about 17% to about 22% by dry weight of Oxalobacter formigenes,
(ii) about 52% to about 62% by dry weight of sucrose;
(iii) about 17% to about 25% by dry weight of one or more cryopreserving agents and/or excipients.

15. The pharmaceutical composition for use according to any one of the preceding claims, wherein said capsule shows essentially no disintegration within one hour of incubation in Simulated Gastric Fluid (SGF) having a pH of about 1.2±0.1 and comprising about 3.2 mg/ml of pepsin at a temperature of about 37° C., but wherein a start of disintegration of said capsule is detected within about one hour in Simulated Intestinal Fluid (SIF) having a pH of about 6.8±0.1 and comprising about 10 mg/ml of pancreatin at about 37° C.

16. The pharmaceutical composition for use according to any of the preceding claims wherein said composition is administered in amounts containing about 10⁹ to about 10¹² CFUs of O. formigenes at least twice a day for a continuous period of time, such as a period lasting for at least months or years, such as 1, 2, 3, 4, 5, 6 or up to 12 months, or 1, 2, 3, 4, or 5 or even more years to a subject in need thereof.

17. A method for the treatment and/or prevention of systemic oxalosis with cardiac involvement in a subject, said method comprising administering a pharmaceutically effective amount of a pharmaceutical composition comprising at least 10⁹ CFUs, such as up to 10¹² CFUs, of viable dried Oxalobacter formigenes present in an enteric-coated capsule, wherein the oxalate-degrading activity in vitro of said Oxalobacter formigenes is no less than 100 mmol oxalate/capsule/19 hours, to a subject in need thereof.

18. The method according to claim 17, wherein said subject is characterized by having a Left Ventricular Ejection Fraction (LVEF) of about ≤55% and/or a Global Longitudinal Strain (GLS) of about ≥−18%.

19. The method according to claim 17 or 18, wherein said subject is on dialysis or will be on dialysis once the treatment is initiated.

20. The method according to any one of claims 17 to 19, wherein said subject is on a stable dialysis regimen throughout the treatment, optionally wherein said subject has been on dialysis at least four months before the treatment is initiated.

21. The method according to any one of claims 17 to 20, wherein as a consequence of an oxalate imbalance in the body of said subject, said subject is on a dialysis treatment.

22. The method according to any one of claims 17 to 21, wherein the treatment or prevention of systemic oxalosis with cardiac involvement in a subject comprises facilitating an improvement in and/or a restoration of a contracting function in the heart muscle of said subject.

23. The method according to any one of claims 17 to 22, wherein said improvement in and/or restoration of a contracting function in the heart muscle is characterized by an improvement in a LVEF and/or a GLS value in said subject, such as when said GLS value in said subject is ≤−18 and/or said LVEF value in said subject is ≥55.

24. The method according to any one of claims 17 to 23, wherein said subject is suffering from end-stage renal disease (ESRD).

25. The method according to any one of claims 17 to 24, wherein said subject is suffering from a Chronic Kidney Disease (CKD) of Stage 5 with risk for heart failure and/or impaired heart elasticity.

26. The method according to any one of claims 17 to 25, wherein said subject has an estimated Glomerular Filtration Rate (eGFR) within the range of 0≤eGFR≤20 ml/min, such as 0≤eGFR≤15 ml/min, 0≤eGFR≤10 ml/min, 0≤eGFR≤5 ml/min, or 0≤eGFR≤0.5 ml/min, when said treatment is initiated.

27. The method according to any one of claims 17 to 26, wherein said subject has had, or will undergo, organ transplantation, such as kidney transplantation.

28. The method according to any one of claims 17 to 27, wherein said pharmaceutical composition comprises:
(i) about 10% to about 25% by dry weight of Oxalobacter formigenes,
(ii) about 50% to about 65% by dry weight of sucrose; and
(iii) about 10% to about 30% by dry weight of one or more cryopreserving agents and/or excipients.

29. The method according to any one of claims 17 to 28, wherein said composition comprises:
(i) about 17% to about 22% by dry weight of Oxalobacter formigenes,
(ii) about 52% to about 62% by dry weight of sucrose;
(iii) about 17% to about 25% by dry weight of one or more cryopreserving agents and/or excipients.

30. The method according to any one of claims 17 to 29, wherein said capsule shows essentially no disintegration within one hour of incubation in Simulated Gastric Fluid (SGF) having a pH of about 1.2±0.1 and comprising about 3.2 mg/ml of pepsin at a temperature of about 37° C., but wherein a start of disintegration of said capsule is detected within about one hour in Simulated Intestinal Fluid (SIF) having a pH of about 6.8±0.1 and comprising about 10 mg/ml of pancreatin at about 37° C.

31. The method according to any one of claims 17 to 30, wherein said composition is administered in amounts containing about 10⁹ to about 10¹² CFUs of O. formigenes at least twice a day for a continuous period of time, such as a period lasting for at least months or years, such as 1, 2, 3, 4, 5, 6 or up to 12 months, or 1, 2, 3, 4, or 5 or even more years to a subject in need thereof.
